(19) United States
(12) Patent Application Publication
(10) Pub. No.: US 2005/0032904 A1
(43) Pub. Date: Feb. 10, 2005

(54) Composition and use of allylamine derivatives

(76) Inventors: Ho Yuan-Soon, Taipei (TW); Lee Wen-Sen, Taipei (TW); Tseng How, Taipei (TW); Chen Chien-Ho, Taipei (TW); Lin Chien-Huang, Taipei (TW); Ho Pei-Yin, Taipei (TW); Chu Jan-Show, Taipei (TW); Ho Wei-Lu, Taipei (TW); Chen Rong-Jane, Tainan (TW); Wang Ying-Jan, Tainan (TW); Jeng Jiiang-Huei, Taipei (TW); Lin Shyr-Yi, Taipei (TW); Liang Yu-Chih, Taipei (TW); Lin Jen-Kun, Taipei (TW); Chen Li-Ching, Hualien (TW)

Correspondence Address: LAW OFFICE OF LIAUH & ASSOC., 4224 WAIALAE AVE, STE 5-388, HONOLULU, HI, 96816, US

(21) Appl. No.: 10/637,931

(22) Filed: Aug. 8, 2003

(51) Int. Cl. A61K 31/137

(52) U.S. Cl. 514/649

(57) ABSTRACT

The present invention discloses the use of allylamine derivatives, such as terbinafine, in the inhibition of cancer cell growth. Also disclosed is the synergistic efficacy of allylamine derivatives in combination with other chemotherapeutically active agents, such as nocodazole, in the inhibition of cancers.

### BACKGROUND OF THE INVENTION

The current options for treating human cancer are limited to excision surgery, general chemotherapy, radiation therapy, and, in aminority of breast cancers that rely on estrogen for their growth, antiestrogen therapy. Although there has been considerable improvement in the treatment of cancer, the overall prognosis remains poor. Cancer remained the leading cause of death, for example, in Taiwan in 2002, for the 21st consecutive year, according to a reviewof the ten leading causes of death in Taiwan in 2002, presented by the Taiwan's Department of Health (DOH). More than a quarter of the disease's victims died from malignant tumors. The five most deadly cancers were liver cancer, lung cancer, cochrane colorectal cancer, breast cancer and stomach cancer. Therefore, investigators continue to search for new therapeutic strategies. One approach seeks to identify medicinal agents capable of retarding the cell cycle and/or activating the cellular apoptotic response in the cancerous cells.

The ability of chemotherapeutic agents to inhibit cancer cell growth and to initiate apoptosis plays an important determinant of their therapeutic response. Another approach attempts to identify combinations of chemotherapeutic agents. Since significant toxicity at high doses has precluded the use of chemotherapeutic agents as a monotherapy for cancers, combination therapy has become one potential method to help reduce undesirable toxic effects of a compound but still maintain or enhance its anti-tumor efficacy.

### SUMMARY OF THE INVENTION

Accordingly, it is an aspect of the invention to provide a compound, its composition, and in combination of other active agents for cancer therapy.

It is another aspect of the invention to provide an allylamine derivative, its composition, and in combination of other active agents for antitumor therapy.

In one aspect, the invention includes a method of treating cancer in warm blooded mammals comprising administering to the mammals a therapeutically effective amount of an allylamine derivative in free base form or in pharmaceutically acceptable salt form.

In other aspect, the invention includes a pharmaceutical composition treating cancer in warm blooded mammals comprising a therapeutically effective amount of the allylamine derivative and a pharmaceutically acceptable carrier.

In another aspect, the invention includes a synergistic pharmaceutical composition for inhibiting the growth of colon cancer, wherein the composition comprises a therapeutically effective amount of the allylamine derivative, the active agent, and a pharmaceutical acceptable carrier.

In one another aspect, the invention includes a method of treating cancer with inducing an anticancer-protein in warm blooded mammals comprising administering to the mammals a therapeutically effective amount of an allylamine derivative in free base form or in pharmaceutically acceptable salt form.

In one another aspect, the invention includes a method of treating cancer with inhibiting a cyclin related protein in warm blooded mammals comprising administering to the mammals a therapeutically effective amount of an allylamine derivative in free base form or in pharmaceutically acceptable salt form. More preferred of the allylamine derivative is terbinafine.

These and other aspects and features of the invention will be more fully appreciated when the following detailed description of the invention is read in conjunction with the accompanying drawings.

### BRIEF DESCRIPTION OF THE DRAWINGS

FIG. 1. shows the dose-dependent effects of TB on cell number in human malignant and normal cells.

FIG. 2. shows time-dependent response of TB-induced G0/G1 phase arrest in COLO 205 cells.

FIG. 3. shows the effects of TB on cell cycle and apoptosis in human cancer cells.

FIG. 4. shows the reversibility of the TB-induced inhibition of cell proliferation.

FIG. 5. shows effects of lower doses (1-5 μM) of TB on cell cycle arrest in human COLO 205 cancer cells.

FIG. 6. shows time effect of TB on cyclin and cdk protein levels in COLO 205 cells.

FIG. 7. shows dose effect of TB on the cell cycle regulatory protein levels.

FIG. 8. shows the amount of cdk4 present in the various cell lines containing different copies of the p53 gene.

FIG. 9. shows TB reduces the growth rate of tumors and potentiates the anti-tumor activity of ND in nude mice.

FIG. 10. shows TB causes the occurrence of apoptosis and increases the levels of p53 and p21/Cip1 protein in the COLO 205-xenografted tumor.

FIG. 11. shows immunolocalization of p53, p21/Cip1, and p27/Kip1 protein in COLO 205 tumor tissues.

### DETAILED DESCRIPTION OF THE INVENTION

Definitions

The term “effective amount” as used herein refers to an amount sufficient to provide an effect sufficient for the inhibition of the growth of tumor cell in vitro and in vivo.

The term “carrier” of “a pharmaceutically acceptable carrier” as used herein refers to a diluent, an excipient, a recipient and the like for use in preparing admixtures of a pharmaceutical composition.

Abbreviations

AS, antisense oligonucleotide
CDK, cyclin-dependent kinase
CKIs, cdk inhibitors
DMSO, dimethylsulfoxide
EMSA, electrophoretic mobility shift assay
FACS, fluorescence-activated cell sorter
FCS, fetal calf serum
I.P., immunoprecipitation
NBT, nitro blue tetrazolium
ND, nocodazole
TB, terbinafine
PMSF, phenylmethyl sulfonyl fluoride
S, sense
SDS-PAGE, sodium dodecyl sulfate-polyacrylamide gel electrophoresis.
   Allylamine Derivatives and its Combination

Recently, the inventors have shown that a number of anti-fungal agents exert antiproliferative and/or apoptotic activities in various malignant cells in vitro and in vivo. For instance, our previous studies showed that ketoconazole induced cell cycle arrest at the G0/G1 phase of the cell cycle and the occurrence of apoptosis in hepatoma and colon cancer cells (Ho YS, et. al., Toxicology & Applied Pharmacology 1998;153(1):39-47; Chen RJ, et. al., Toxicology & Applied Pharmacology 2000;169(2):132-41.), whereas griseofulvin induced apoptosis and cell cycle arrest at the G2/M phase through abnormal microtubule polymerization (Ho YS, et. al., International Journal of Cancer 2001;91(3):393-401.).

One of the well known anti-fungal agent is allylamines derivatives. Preferable examples of allylamines class include the following products:

(1) Terbinafine, 1-Naphthalenemethanamine, N-(6,6-dimethyl-2-hepten-4-ynyl)-N-methyl-, (E)-, an antimycotic allylamine, or turbinefine hydrochloride.

(2) Naftifine, 1-Naphthalenemethanamine, N-methyl-N-(3-phenyl-2-propenyl)-(E).

(3) Butenafine, N-(p-tert-Butylbenzyl)-N-methyl-1-naphthalenemethylamine, a benzyl amine antifungal, or butenafine hydrochloride. Most preferred of the allylamines derivatives is nocodazole,

TB, is a newly synthesized oral antimycotic drug in the allylamines class, and a fungicidal agent that inhibits ergosterol synthesis at the stage of squalene epoxidation. (Petranyi G, et. al., Science 1984; 224(4654): 1239-41.). It shows a good safety profile and relatively few drug interactions (Abdel-Rahman SM, et. al., Annals of Pharmacotherapy 1997;31(4):445-56.). The cream form and oral tablet of TB have been approved for clinical uses in the United States. (Gupta A K, et. al., Journal of the American Academy of Dermatology 1997;37(6):979-88.). The oral formulation has been on the market in various countries for more than eight years, and as of 1997, more than 7.5 million individuals had been treated with this drug (Gupta A K, et. al., Clinical & Experimental Dermatology 1998;23(2):64-7.). The anti-tumoral activity of TB was examined by this invention.

Furthermore, our previous research demonstrated that griseofulvin, an oral antifungal agent, potentiates the anti-cancer activities of nocodazole, ND, a clinical used chemotherapeutic agent, in vivo. (Ho YS, et. al., International Journal of Cancer 2001; 91(3):393-401.). Although any chemotherapeutic agent can be included in the present invention, preferred chemotherapeutic agents include those selected from the group consisting of chemotherapeutic agents albendazole, fenbendazole, nocodazole, parbendazole, mebendazole, oxibendazole, carbendazim, thiabendazole and benzimidazole, and combinations thereof. Most preferred of the chemotherapeutic agents is nocodazole, ND. ND is also one of the natural and synthetic epothillones, including but not limited to epothillones of A, B, C, and D. For searching more methods to treat cancers, the inventors also showed that combined treatment of griseofulvin and nocodazole significantly enhanced the therapeutic efficacy in the treatment of cancerous cells in athymic mice bearing COLO 205 tumor xenografts. (Ho YS, et. al., International Journal of Cancer 2001; 91(3):393-401.). In this invention, consequently, an enhancement of TB on the ND-induced apoptosis was further demonstrated.

Inhibition of Cell Proliferation in TB(teribinafine)—Treated Human Malignant Cells

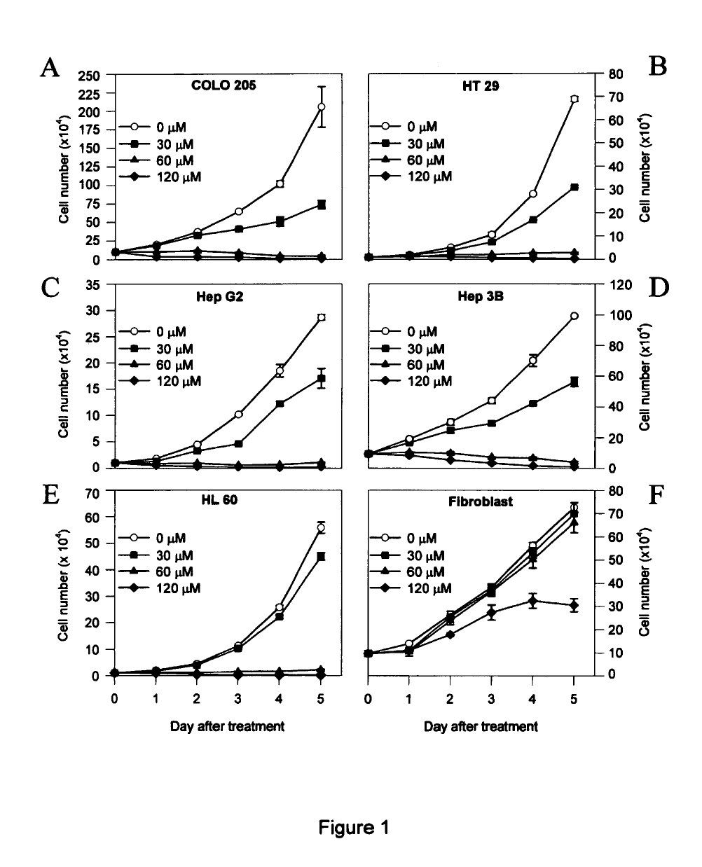

The effect of TB on the growth of various human cancer cells wasexamined. In FIG. 1, (A) COLO205, (B) HT29, (C) HepG2, (D) Hep3B, (E) HL60, and (F) normal human fibroblast cells were treated with various concentrations of TB (30 to 120 μM). Media with or without TB were renewed daily until cell counting. Three samples were analyzed in each group. Values represent mean±S.E. The cells were cultured for 5 days with or without TB (30-120 μM), and then the cells were harvested and counted. These data show that TB decreased cell number in cultured human cancer cells (COLO 205, HT29, HepG2, Hep3B and HL60) in a dose-dependent manner. When TB concentration was increased to 60 μM, cell growth arrest or cell death was observed in these cancer cells. In contrast, TB at concentrations of 30-60 μM did not inhibit the growth rate of the cultured human gingival fibroblasts (FIG. 1F). However, when TB concentration was increased to 120 μM, a 50% growth inhibition was observed.

Arrest of Cell Cycle at the G0/G1 Phase by TB in Human Cancer Cells

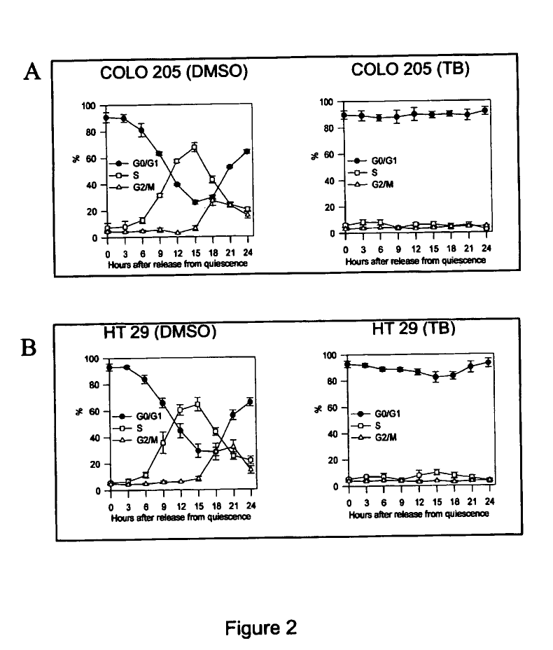

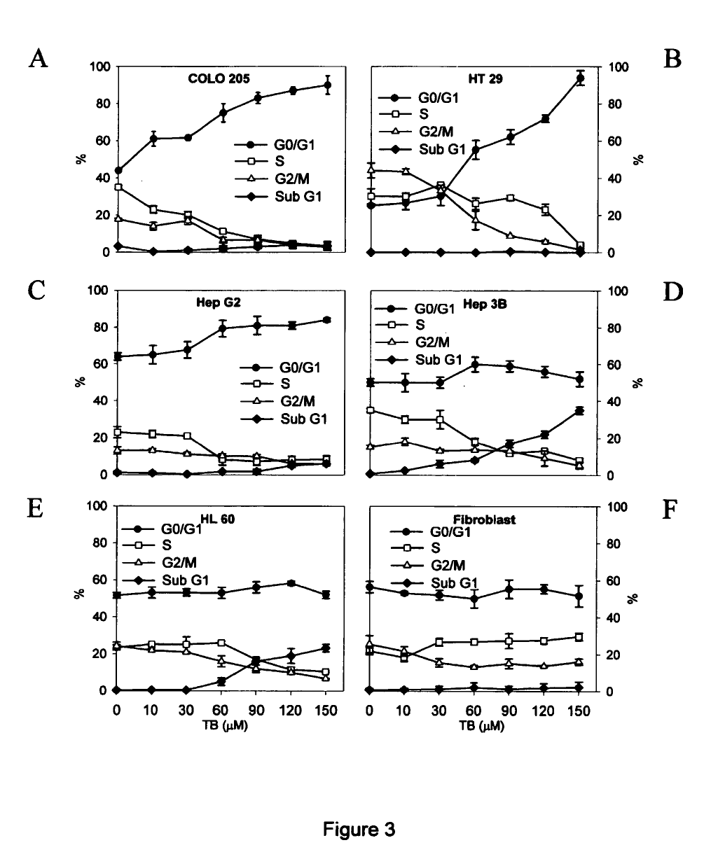

In order to distinctively demonstrate the actions of TB on a specific phase of the cell cycle, the cancer cells (COLO 205 and HT 29) were all synchronized by switching them to media with 0.04% FCS for 24 h to render them quiescent. When they were returned to culture media containing 10% FCS and 0.05% DMSO (control) or 90 μM TB in 0.05% DMSO (which started them all on a new cell cycle) and, at various times thereafter, they were harvested for flow cytometry analysis. FIG. 2 shows the time-dependent response of TB-induced G0/G1 phase arrest in COLO 205 and HT 29 cells. COLO 205 (A) and HT29 (B) cells were synchronized with 0.04% FCS for 24 h as described in Examples. After synchronization, the cells were then released into complete medium (10% FCS) containing 0.05% DMSO (left panel), or 90 μM TB in 0.05% DMSO (right panel). Percentage of cells in G0/G1, S, and G2/M phases of the cell cycle were determined using established CellFIT DNA analysis software. Three samples were analyzed in each group, and values represent the mean±S.E. In FIG. 2, it further shows the representative FACS analyses of DNA content of the DMSO-(left panel) and the 90 μM TB-(right panel) treated COLO 205 (FIG. 2A) and HT 29 (FIG. 2B) cells at various times after the cells release from quiescence. The results demonstrate that TB induced an accumulation (>90%) of the COLO 205 and HT 29 cells at the G0/G1 phase of the cell cycle, suggesting that the observed growth inhibitory effect of TB on the COLO 205 and HT 29 cells was due to an arrest of G0/G1 phase in the cell cycle. FIG. 3 demonstrated the dose effect of TB on the G0/G1 arrest and shows TB dose-dependently induced cell cycle arrest at the G0/G1 phase in COLO 205 (A), HT29 (B) and HepG2 (C). In Hep3B (D) and HL60 (E), TB caused the occurrence of apoptosis in a dose-dependent manner. Treatment of human fibroblast for 15 h did not cause cell cycle arrest or apoptosis (F). FACS analysis of DNA content after 15 h release from quiescence by incubation in culture media supplemented with 10% FCS and various concentrations of TB in 0.05% DMSO. Percentage of cells in G0/G1, S, and G2/M phases of the cell cycle were determined using established CellFIT DNA analysis software. Three samples were analyzed in each group, and values represent the mean±S.E. As illustrated in FIGS. 3A-C, TB induced G0/G1 arrest in COLO 205, HT 29 and Hep G2 cells in a dose-dependent manner. In Hep 3B and HL 60 cells (p53 null), however, TB (10-150 μM) did not induce G0/G1 arrest, but dose-dependently caused the occurrence of apoptosis as evidenced by the presence of the sub G1 (FIGS. 3D and E). Importantly, treatment of human fibroblasts with TB did not induce cell cycle arrest or cell death (FIG. 3F)

TB-Induced G0/G1 Arrest is Irreversible

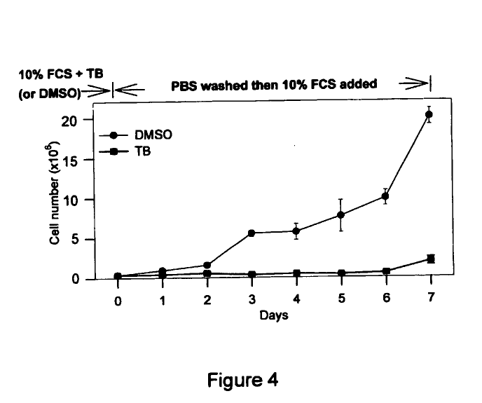

The reversibility of the TB-induced G0/G1 arrest was tested by removing TB after induction of cell cycle arrest. FIG. 4 shows TB-induced inhibition of cell proliferation was not reversed by removal of TB. The COLO 205 cells were released from quiescence by incubation in culture media supplemented with 10% FCS and 0.05% DMSO without or with 90 μM TB for 24 h. After 24 h treatment with TB, the cells were washed twice with PBS, replaced with fresh 10% FCS without DMSO or TB. The TB-induced inhibition of cell proliferation was sustained for at least 7 days after removal of TB. The cultured cell numbers in DMSO- and TB-treated group were counted every day. Three samples were analyzed in each group, and values represent the mean±S.E.

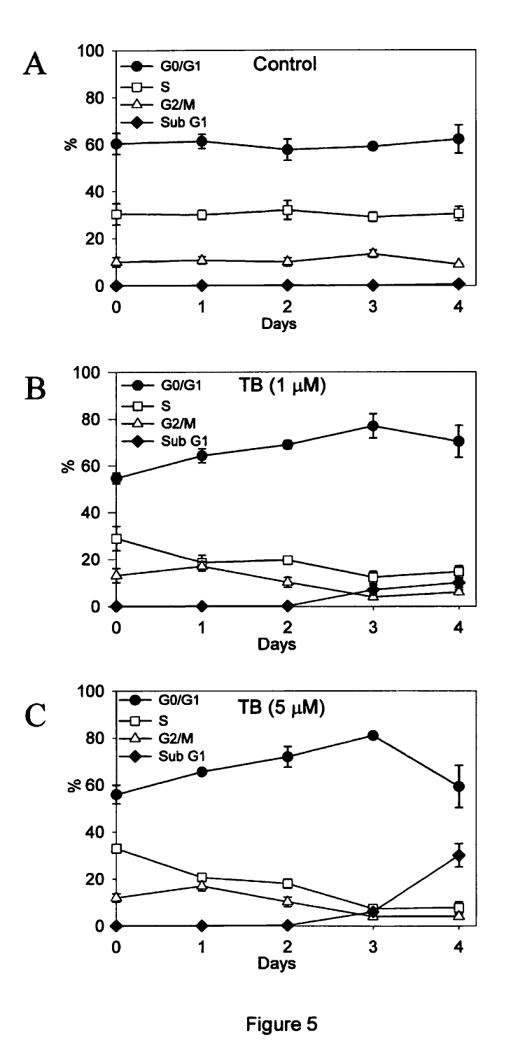

The outcome in FIG. 4 demonstrated that the TB-induced G0/G1 cell cycle arrest was not reversed by removal of TB, and this inhibition lasted for at least 7 days. The invention further tested whether TB can induce cell cycle arrest at a lower concentration with longer exposure. As illustrated in FIG. 5, treatment of COLO 205 with TB at a concentration as low as 1 μM for 4 days can induce significant G0/G1 cell cycle arrest. TB dose- and time-dependently induced cell cycle arrest at the G0/G1 phase in COLO 205 cells. The COLO 205 cells were exposed to culture media supplemented with 10% FCS and various concentrations (1 and 5 μM) of TB in 0.05% DMSO for the indicated times. Media with and without TB were changed daily until flowcytometry analysis performed. Percentage of cells in G0/G1, S, and G2/M phases of the cell cycle were determined using established CellFIT DNA analysis software. Three samples were analyzed in each group, and values represent the mean±S.E.

Effects of TB on the Levels of Cell Cycle Regulatory Proteins

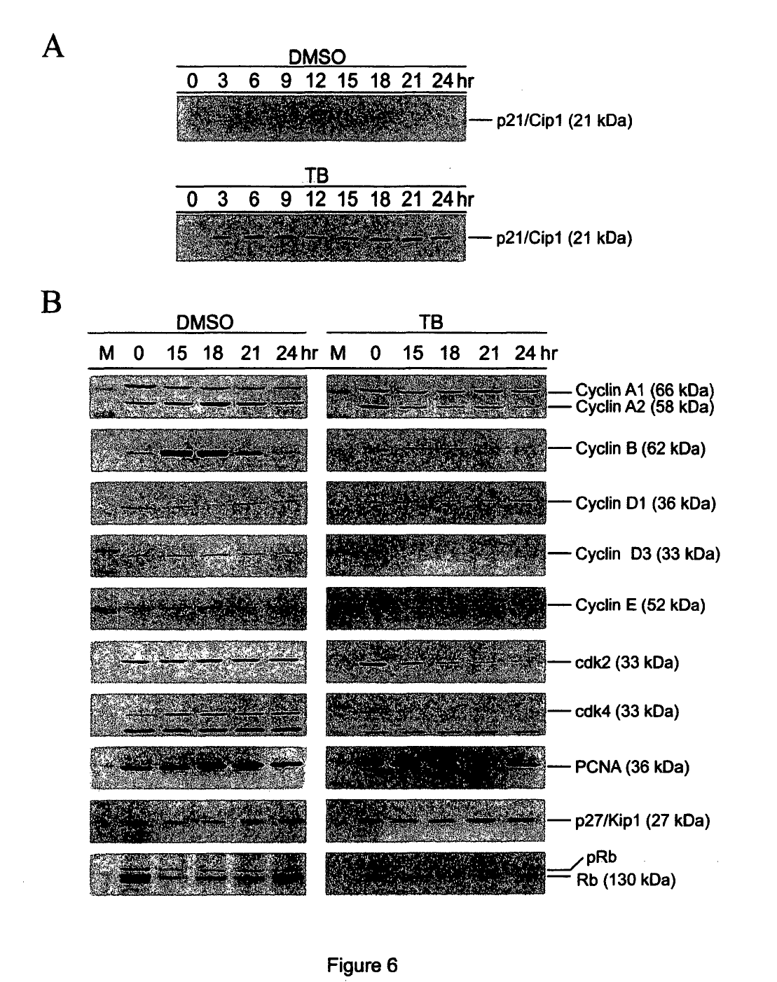

To investigate the underlying molecular mechanisms of TB-induced G0/G1 arrest, the COLO 205 cells were switched to media with 0.04% FCS to render them quiescent at the G0/G1 phase. They were then returned to culture media supplemented with 10% FCS and 0.05% DMSO with or without TB (60 μM), and at various times thereafter, they were harvested for protein extraction and Western blot analysis. Based on the FACS analysis in the COLO 205 cells, 0, 15, 18 and 24 h after release from quiescence represents the G0/G1, S, G2/M and 2nd G0/G1 phase of the cell cycle, respectively. (FIG. 2A). Accordingly, these time points were selected for protein extraction and Western blot analysis to examine the effects of TB on the expression of cell cycle regulatory proteins. In FIG. 6, (A) In the DMSO-treated COLO 205, 10% FCS caused a transit increase in p21/Cip1 protein (upper panel). In contrast, TB caused a persistent increase in p21/Cip1 protein level (lower panel). (B) In response to TB treatment, the levels of cyclin A2, B and D3, cdk2, cdk4, and pRB protein were downregulated, while the cyclin. E and p27/Kip1 were slightly upregulated. The COLO 205 cells were synchronized with 0.04% FCS for 24 h, and then released into complete medium (10% FCS) containing TB (60 μM) for the indicated time points. COLO 205 cells were also treated with DMSO (0.05%, v/v) as a control group. Protein extracts (100 μg/lane) were separated by SDS-PAGE, probed with specific antibodies, and detected using the NBT/BCIP system. As shown in the FIG. 6A (upper panel), the level of p21/Cip1 protein in the DMSO-treated COLO 205 cells was increased significantly at 3 h after the cells were challenged with 10% FCS, and then rapidly declined to an undetectable level at 9 h after treatment. This result was consistent with a previous report showing that transient induction of p21/Cip1 was required for the increased stability of cdk kinases activity. (LaBaer J, et. al., Genes & Development 1997;11(7):847-62.). The TB-treated COLO 205 cells, on the other hand, showed a persistent increase in p21/Cip1 protein level after TB treatment (FIG. 6A, lower panel).

A previous study showed that the p27/Kip1 protein level was high in the quiescent cells, and then decreased rapidly after stimulated with serum. (Coats S, et. al., Science 1996;272(5263):877-80.). Similar finding was observed in FIG. 6B showing that the level of p27/Kip1 protein in the COLO 205 cells was high after 24 h serum starvation (left panel, 0 h), and then decreased after challenged with 10% FCS (left panel, 15 h). In contrast, the increased p27/Kip1 protein levels in the TB-treated COLO 205 cells were maintained at high levels after 10% FCS treatment (right panel, 0-24 h). The levels of cyclin D3, cdk2 and cdk4 protein were downregulated in the TB (60 μM)-treated COLO 205 cells, while the levels of cyclin D1 and PCNA protein were not changed (FIG. 6B, right panel). Cyclin A2 and cyclin B, which promoted the cell entrance into the S and the G2/M phase respectively, were also downregulated in the TB-treated COLO 205 cells (FIG. 6B, right panel). The levels of cyclin E protein, which is associated with cdk2, were slightly elevated in the TB-treated cells (FIG. 6B, right panel). Moreover, the levels of phosphorylated Rb (pRb) were dowuregulated in the TB-treated COLO 205 cells.

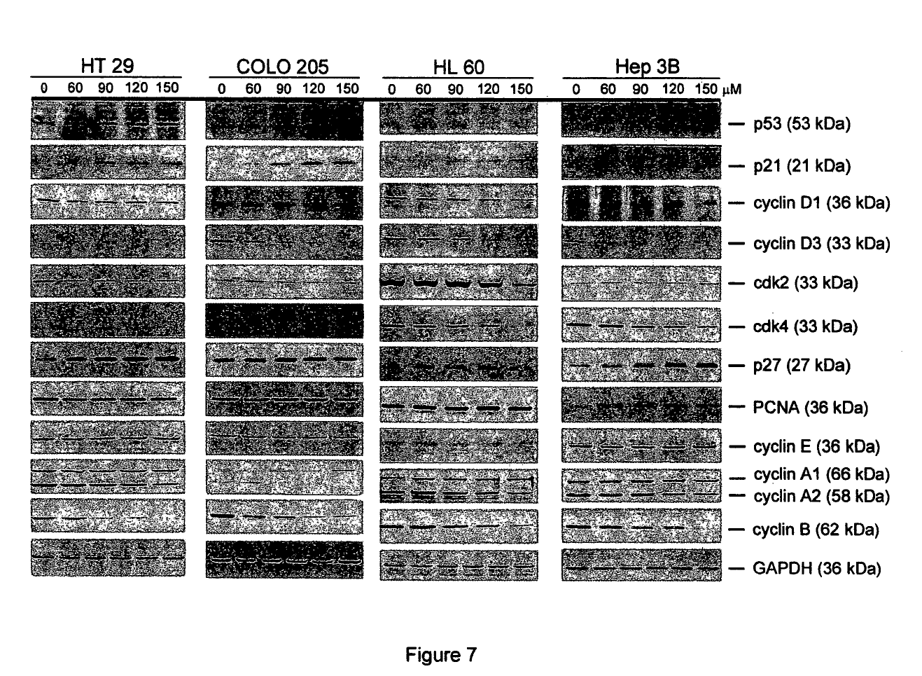

The results shown in FIG. 3 demonstrated that TB induced cell cycle arrest at the G0/G1 phase in human cancer cells with either wild-type p53 (COLO 205 and HepG2) or p53 His²⁷³ mutant (HT 29). In contrast, TB induced apoptosis in the HL 60 (p53 null) and Hep 3B (p53 partial deleted). These data suggest that TB induced the cancer cells to undergo G0/G1 cell cycle arrest or apoptosis dependent on the p53 status of the cells. To further test this hypothesis, the dose effects of TB on the levels of cell cycle regulatory protein were conducted in four different human cancer cell lines, COLO 205 (p53 wild type), HT 29 (p53 His²⁷³ mutant), Hep 3B (p53 partial deleted) and HL 60 (p53 null). As illustrated in FIG. 7, TB increased the levels of p53, p21/Cip1 and p27/Kip1 protein, and decreased cyclin D3 and cdk4 in COLO 205 and HT 29 cells. In HL 60 and Hep 3B cells, TB treatment did not change the levels of p53 and p21/Cip1 protein, but significantly increased the levels of p27/Kip1 protein. TB dose-dependently increased the levels of p53, p21/Cip1 and p27/Kip1 protein, and decreased the levels of cyclin D3 and cdk4 protien in COLO 205 and UT 29 cells. In HL 60 and Hep 3B cells, TB treatment did not change the levels of p53 and p21/Cip1 protein, but significantly increased the levels of p27/Kip1 protein in a dose-dependent manner. The cells were rendered quiescent for 24 h, and then challenged with 10% FCS and treated with various concentrations of TB (60-150 μM) for additional 15 h. Protein extracts (100 μg/lane) were separated by SDS-PAGE, probed with specific antibodies, and detected using the NBT/BCIP system. Membranes were also probed with anti-GAPDH antibody to correct for differences in protein loading.

P53 Activated Signaling Pathway was Involved in TB-Induced G0/G1 Arrest

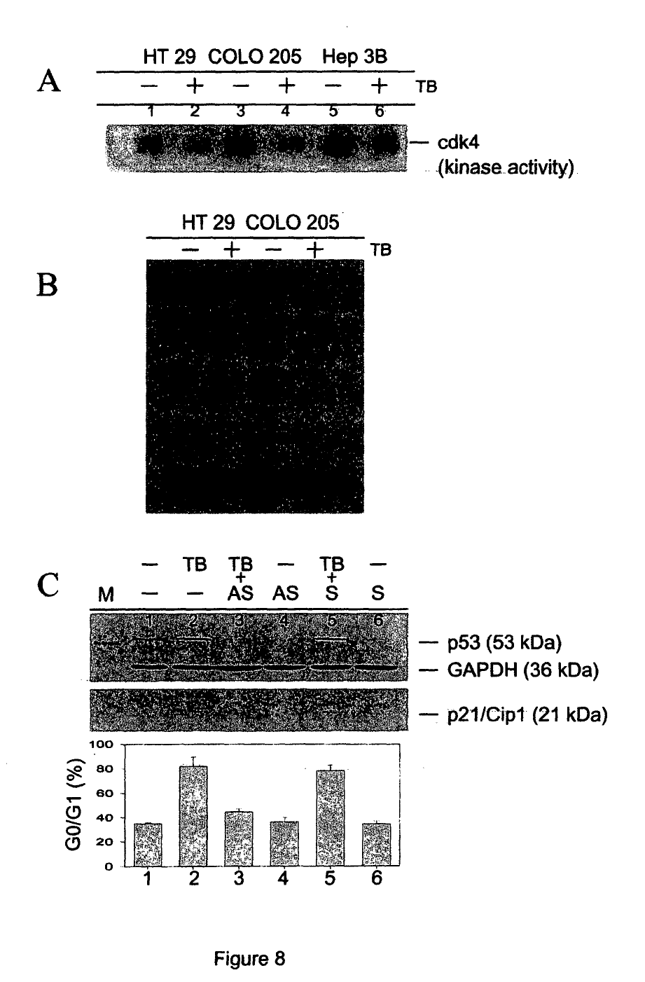

The p53 protein has been suggested to be a potent transcription factor involved in the regulation of cell cycle arrest and occurrence of apoptosis. (Ko U, and Prives C. Genes & Development 1996; 10(9): 1054-72; Levine AJ., Cell 1997;88(3):323-31.). As illustrated in FIG. 7, the levels of p21/Cip1 and p53 protein were dose-dependently increased in TB-treated COLO 205 and HT 29 cells, suggesting that upregulation of p53 and p21/Cip1 might be involved in the TB-mediated G0/G1 arrest in these cells. To further test this hypothesis, we examined the TB effects on the levels of p21/Cip1, p27/Kip1 and p53 protein, and the cdk4 kinase activity in three human cancer cell lines (COLO 205, HT 29 and Hep 3B). FIG. 8 shows: (A) TB strongly decreased the assayable cdk4 kinase activity in COLO 205 cells (p53 wild type). In the HT 2.9 (p53 His²⁷³ mutated) and Hep 3B cells (p53 partial deleted), TB slightly decreased the cdk4 kinase activity. The cells were treated with 60 μM TB (+) or 0.05% DMSO (−) for 15 h after released from quiescence; (B) Treatment of COLO 205 cells, but not HT 29 cells, with 60 μM TB increased the binding between p53 protein and p53 consensus binding site in p21/Cip1 promoter DNA probe; and (C) Antisense p53 oligonucleotide abolished the TB-mediated increases of p53 and p21 protein levels and cell population at the G0/G1 phase. Antisense or sense p53 was added to COLO 205 at a final concentration of 20 μM at 16 h before the cell was challenged with 10% FCS and 60 μM TB treatment for additional 15 h. Percentage of cells in G0/G1 phase of the cell cycle determined using established CellFIT DNA analysis software is shown in the bottom. The symbols of “M,” refers to “size marker;” “AS,” to “antisense p53 oligonucleotide;” and “S,” to “sense p53 oligonucleotide.” As shown in FIG. 8A, TB at a concentration of 60 μM induced a strong decrease in the assayable cdk4 kinase activity in COLO 205 (p53 wild type) cells, and a slight decrease in the TB-treated HT-29 (p53 His273 mutant) and Hep 3B (p53 partial deletion) cells. The electrophoretic mobility gel shift assay was conducted by using p21/Cip1 promoter DNA, which contains p53 consensus binding site, to demonstrate that the p53 binding activity was increased more significantly in the nuclear extracts of the TB-treated COLO 205 cells (FIG. 8B, lane 4), than in those of the TB-treated HT 29 cells (FIG. 8B, lane 2).

To further demonstrate that in the TB-treated cells, increased p53 expression correlated with G0/G1 arrest, the experiment illustrated in FIG. 8C was carried out. Thus, in the sample labeled TB (for 60 μM TB-treated alone), the p53 and p21 electrophoretogram bands were increased in intensity, while G0/G1 population was increased by about 2.3 fold (FIG. 8C, lane 2). Sample TB+AS was treated with a p53 antisense oligonucleotide (AS), which blocked the expression of p53. Consequently, in this sample, the levels of p53 and p21 protein did not increase and the TB addition to sample TB+AS failed to induce the increased G0/G1 population, which was evident in the TB sample (FIG. 8C, lane 3).

TB Potentiates the Apoptotic Effects of Nocodazole (ND)

Combined treatment of the cells with drugs affecting different cell cycle checkpoints has been suggested to be one of the approaches to enhance the drug-induced apoptotic effect in human malignant cells. (Li CJ, et. al., Proceedings of the National Academy of Sciences of the United States of America 1999; 96(23): 13369-74.).

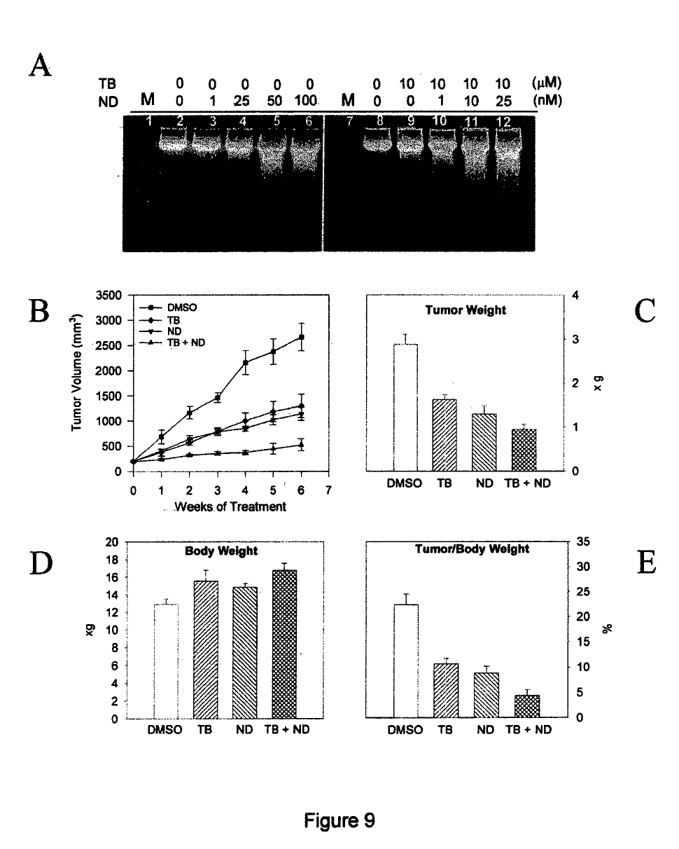

Accordingly, we co-treated the COLO 205 cells with TB, which causes G0/G1 arrest, and ND, which arrests the cells at the G2/M phase, and examined the degree of the occurrence of apoptosis. Genomic DNAs extracted from TB-treated COLO 205 were examined by gel electrophoresis. They were found to display the DNA ladder patterns characteristic of cells undergoing apoptosis when ND was at a concentration of 50 nM or higher (FIG. 9A, lane 5). In FIG. 9, the part of (A) refers to potentiation of ND-induced apoptosis by TB. Induction of apoptosis in COLO 205 cells was shown by DNA fragmentation using electrophoresis of genomic DNA. DNA fragmentation was examined at 24 h after drug treatment. In the lanes 2 and 8, cells received mock treatment as controls. The symbols of “M” (in lanes 1 and 7), refer to “DNA size marker.” (B) average tumor volume of DMSO-treated vs. drug-treated nude mice (n=5). (C) tumor weight, (D) animal body weight, and (E) tumor/body weight ratio were measured at the end of experiment. Five samples were analyzed in each group, and values represent the mean±S.E. Comparisons were subjected to ANOVA followed by Fisher's least significant difference test. Significance was accepted at p<0.05. *TB-, ND-, and TB+ND-treated group different from DMSO-treated group. TB+ND-treated group different from TB- or ND-treated group. In the presence of 10 μM TB, which does not induce DNA ladder in COLO 205 cells (FIG. 9A, lane 9), ND induced the DNA ladder pattern in the COLO 205 cells at a concentration as low as 1 nM (FIG. 9A lane 10). This finding indicates that TB enhanced the ND-induced apoptosis in the COLO 205 cells.

Given the enhancement by TB of the ND-induced apoptosis of COLO 205 cells in vitro, we next determined whether administration of TB could affect the ND-induced obvious decline in tumor size in an in vivo setting. A reduction in tumor volume between mice given TB, ND or TB plus ND vs. those given vehicle (DMSO plus peanut oil) was detected (FIGS. 9B-E). Importantly, the tumor volume and tumor weight of the mice treated with TB plus ND were significantly reduced as compared with those treated with TB or ND alone (p<0.05), suggesting that TB enhanced the ND-induced reduction in tumor size.

Both Cell Cycle Arrest and Occurrence of Apoptosis are Involved in the TB-Inhibited Tumor Growth In Vivo

Since, retardation of the cell cycle and/or activation of the cellular apoptotic response are two major mechanisms preventing tumor growth, we examined the TB effect on the cell cycle and apoptosis occurrence of the solid tumor derived from the implanted COLO 205. The COLO-205 cells (5×10⁶) in 0.1 ml RPMI 1640 were injected subcutaneously between the scapulae of each nude mouse (purchased from National Science Council animal center, Taipei). After transplantation, the tumor size was measured using calipers and the tumor volume was estimated by the formulae: tumor volume (mm³)=1/2×L×W², where L is the length and Wisthewidthofthetumor. (OkiE, et. al., British Journal of Cancer 1998;78(5):625-30.). Once the tumor reached a volume of 200 mm³, animals received intraperitoneal injections of DMSO (25 μl), TB (50 mg/kg), ND (5 mg/kg), or TB plus ND 3 times per week for 6 weeks.

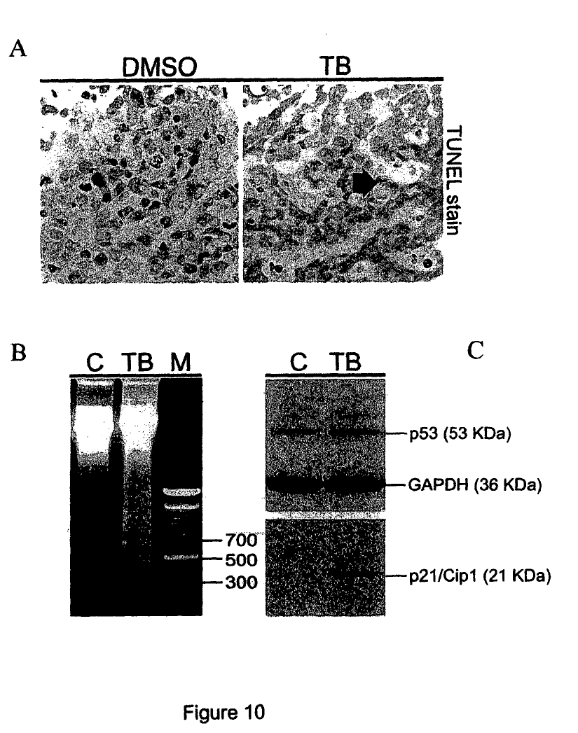

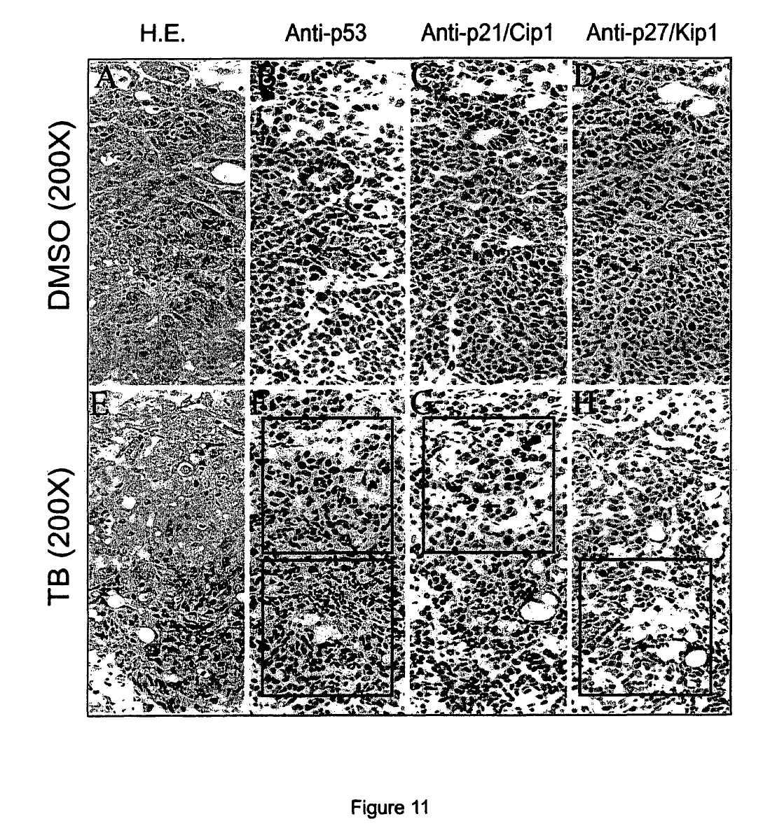

In FIG. 10, light micrographs of COLO 205 tumor tissues stained in situ by the TdT-mediated dUTP-biotin nick end labeling method to detect the DNA breaks (A) (400×). Arrow indicates representative apoptotic cells. Induction of apoptosis by TB in COLO 205 tumor is also shown by DNA fragmentation using electrophoresis of genomic DNA (B). Western blot analyses show the levels of p53 and p21/Cip1 protein in COLO 205 tumor (C). COLO 205 tumors were isolated for protein extraction at 6 weeks after DMSO or TB treatment. Membranes were also probed with anti-GAPDH antibody to correct for differences in protein loading. The particular evidence for the occurrence of apoptosis in the tumor isolated from the TB-treated animal includes DNA strand breaks caused by endonuclease, which can be detected in situ by nick end labeling tissue sections with dUTP-biotin by terminal deoxynucleotidyl transferase (FIG. 10A), and fragmentation of DNA, which can be examined by gel electrophoresis (FIG. 10B). The contents of p53 and p21/Cip1 were increased in the tumor isolated from the TB-treated mouse (FIG. 10C), suggesting that the inhibition of the progression of cell cycle activity was involved in the TB-induced decline in tumor size. The TB-induced upregulations of p21 and p53 in the COLO 205 tumor were further confirmed by immunocytochemical staining technique. In FIG. 11, strong immunoreactivities of p53 (D, blue and red square), p21/Cip1 (E, blue square) and p27/Kip1 (F, red square) protein were detected in COLO 205 tumor tissues isolated from the TB-treated nude mice, but not from the DMSO-treated mice (A-C). The tumor tissues were cut into 5-7 μm thickness and serial sections were stained with the specific antibodies against the human p53 (A and D), p21/Cip1 (B and E), and p27/Kip1 (C and F) for determination of specific antigen in tumor tissues. Green arrows indicate the representative p53 (D), p21/Cip1 (E), or p27/Kip1 (F) immunoreactive cells (brown) (200×). The percentage of cells expressing p53 (G), p21/Cip1 (H) and p27/Kip1 (I) were calculated. The DMSO-treated animals (control) expressed very little, if any, p53, p21/Cip1 and p27/Kip1 activity in the COLO 205 tumor tissue (FIGS. 11A-C). In contrast, p53, p21/Cip1 and p27/Kip1 immunoreactivities were strongly induced in the TB-treated tumor tissues (FIGS. 11D-F). Interestingly, the p53 immunoreactive cells were observed in all over the whole tissue section. Among these cells, some expressed p21/Cip1 and the others expressed p27/Kip1. The p21/Cip1 (FIG. 11E, blue square) and p27/Kip1 (FIG. 11F, red square) immunoreactive cells located in different areas of the COLO 205 tumor tissues. FIGS. 11G-I, and I showed the percentage of cells expressing p53, p21/Cip1 and p27/Kip1 respectively.

Discussion

The present study was undertaken to investigate the anti-cancer mechanisms of TB. Our in vitro studies demonstrated that TB inhibited proliferation and induced apoptosis in cultured human cancer cells. In vivo studies showed that intraperitoneal administration of TB at a dose of 50 mg/kg caused a substantial decline in tumor size of the COLO 205 tumor mass. An increased expression of p21/Cip1 and p53 protein and the occurrence of apoptosis in the solid tumor isolated from the TB-treated mouse suggest that both cell cycle inhibition and apoptotic cell death contribute to the anti-tumor effects of TB. To our knowledge, this is the first demonstration that TB inhibits the growth of colon cancer cells both in vitro and in vivo through retardation of the cell cycle and activation of the cellular apoptotic response in the cancer cells.

The inhibitory effect of TB on cell growth does not appear to be limited to the COLO 205 cells, as similar inhibition has also been observed in other transformed cultured cells, such as HT29, HepG2, Hep3B and HL60 (FIG. 1). However, it seems that TB exerts its anti-tumor activity through retarding the cell cycle or activating the cellular apoptotic response dependent on the p53 status of the cancer cells. In this report we observed that TB dose-dependently induced cell cycle arrest at the G0/G1 phase in the cancer cells with wild-type p53 (COLO 205 and Hep G2) and the occurrence of apoptosis in the cells with p53 null (HL 60) or partial deletion p53 (Hep 3B). Previous report demonstrated that HT-29 cells contain a point mutation at codon 273 (Arg→His) of the p53 gene. (Niewolik D, et. al., Oncogene 1995;10(5):881-90.). In the HT-29 cell, TB treatment caused cell cycle arrest instead of apoptosis (FIG. 3B). This result is consistent with a recent report showing that mutant p53 was not sufficient to induce apoptosis. (Rowan S, et. al., EMBO Journal 1996;15(4):827-38.). The mutant-type p53 protein in HT-29 cells is recognized by pAb DO-1 (recognizes all p53 proteins) and pAb 1620 (recognizes wild-type conformation of p53) but not by pAb 240 (recognizes mutant conformation of p53). (Webley K M, et. al., Journal of Pathology 2000;191(4):361-7.). In this study, the mutant-type p53 protein in HT-29 cells (with wild-type p53 conformation) could be detected with pAb 1620. However, the degree of p53 induction by TB in HT-29 cells was much less than that observed in COLO 205 cells (FIG. 8A). These observations demonstrated that both of the COLO 205 cells, which express wild-type p53, and the HT 29 cells with wild-type p53 were sensitive to TB-induced G0/G1 arrest. Such results implied that p53 signaling pathway was involved in the G0/G1 cell cycle arrest.

Treatment of the COLO 205 cells with TB resulted in an increase in the levels of p21/Cip1, p27/Kip1 and p53 protein and a decrease in the levels of cyclins A2, B, and D3, cdk2 and cdk4 protein (FIGS. 6 and 7). Among these changes, p53 seems to have a major contribution to the TB-induced G0/G1 arrest in the COLO 205 cells. P53, the tumor suppressor, has been implicated in a variety of cellular processes. (Greenblatt M S, et. al., Cancer Research 1994;54(18):4855-78; Bates S, and Vousden KU. Current Opinion in Genetics & Development 1996;6(1): 12-8.). However, the undisputed roles of p53 are the induction of cell growth arrest and apoptosis. (el-Deiry W S, et. al., Cancer Research 1994;54(5):1169-74; el-Deiry W S, et. al., Cell 1993;75(4):817-25.). In response to TB treatment, the expression of p53 in COLO 205 and HT 29 were significantly upregulated. The TB-induced increase of p53 did bind to the p21/Cip1 promoter DNA, which contains p53 consensus binding site (FIG. 8B). In this study, we further demonstrated that the process of G0/G1 cell cycle arrest induced by TB in the COLO 205 cells is correlated with the activation of the p53-associated signaling pathway, as evidenced by the p53 specific anti-sense oligonucleotide experiment (FIG. 8C). Moreover, TB-induced G0/G1 arrest was not observed in the p53 null cells, HL 60 (FIG. 3E). These data further support the notion that p53 is involved in the TB-induced antiproliferaton. Observation of an increased expression of p53 and p21/Cip1 proteins and the occurrence of apoptosis in the solid tumor isolated from the TB-treated mouse supports the hypothesis that the p53-signaling pathway is involved in TB-induced decline in tumor size of the COLO 205 tumor. An increased expression of p21/Cip1 protein and a decreased of the assayable cdk4 kinase activity (FIG. 8A) in the TB-treated COLO 205 cells suggests that TB treatment caused an increase in p53 protein level, which in turn upregulated the p21/Cip1 level, and finally induced a decrease in the cdk kinase activity. The consequent reduction of cdk4 activity by p21/Cip1 is most likely responsible for the TB-induced G0/G1 arrest in the COLO 205 cells.

In this study, we try to clarify the roles of the p21/Cip1 and p27/Kip1 protein expression, which involved in G0/G1 arrest and (or) apoptosis induced by TB. Previous studies have demonstrated that p21/Cip1 arrests the cell cycle through binding and inactivating the cdks, which are required for cell cycle progression. (el-Deiry W S, et. al., Cancer Research 1994;54(5): 1169-74; el-Deiry W S, et. al., Cell 1993;75(4):817-25.). A number of studies have suggested that p21/Cip1 does have tumor suppressor properties. P21/Cip1 mutations have been found in several human tumors (Malkowicz S B, et. al., Oncogene 1996;13(9):1831-7.); and a p21/Cip1 mutation, which was demonstrated to specifically abrogate its binding to cdks, was identified in a primary breast tumor. (Balbin M, et. al., Journal of Biological Chemistry 1996;271(26):15782-6.). P27/Kip1 also mediates growth arrest and is thought to play a critical role in negative regulation of cell division in vivo. (Naumann U, et. al., Biochemical & Biophysical Research Communications 1999;261(3):890-6.). In contrast to p21/Cip1, mice with the p27/Kip1 gene null showed an increased body size, female sterility and a high incidence of spontaneous pituitary tumors. (Fero M L, et. al., Cell 1996;85(5):733-44.). In this study, we demonstrated that the p27/Kip1 was significantly induced by TB in both HL60 (p53 null) and Hep3B (p53 deleted) cells (FIG. 7). However, significant G0/G1 phase cells cycle arrest in both of the Hep3B and HL60 cells were not observed (FIGS. 3D and E). Recent study demonstrated that adenovirally mediated p27/Kip1 overexpression leads to apoptosis in human cancer cells. In sharp contrast, a similar over expression of p21/Cip1 results in G1-S arrest but minimum cytotoxicity was observed. (Katayose Y, et. al., Cancer Research 1997;57(24):5441-5.). Another study demonstrated that p27/Kip1 expression was associated with spontaneous apoptosis and Bax protein expression in tumor sections from oral and orthopharyngeal carcinoma in vivo and in vitro. (Fujieda S, et. al., International Journal of Cancer 1999;84(3):315-20; Wang X, et. al., Oncogene 1997;15(24):2991-7.). All these results implied that p27/Kip1 protein might play an important role in TB-induced apoptosis but not in cell growth arrest.

The ability of chemotherapeutic agents to inhibit cancer cell growth and to initiate apoptosis plays an important determinant of their therapeutic response. However, significant toxicity at high doses has precluded the use of chemotherapeutic agents as a monotherapy for cancers. Combination therapy is one potential method to help reduce a compound's undesirable toxic effects but still maintains or enhance its anti-tumor efficacy. Recently, our study demonstrated that griseofulvin, an oral antifungal agent, potentiates the anti-cancer activities of ND, a clinically used chemotherapeutic agent, in vivo. (Ho YS, et. al., International Journal of Cancer 2001; 91(3): 393-401.). In the present study, we further demonstrated an enhancement of TB on the ND-induced apoptosis. Historically, TB has been used as an orally active broad-spectrum antifungal drug, especially active in patients with histoplasmosis or nonmeningeal cryptococcosis. (Rademaker M, and Havill S. New Zealand Medical Journal 1998; 111(1060):55-7; Caceres-Rios U, et. al., Journal of the American Academy of Dermatology 2000;42(1 Pt 1):80-4.). A previous study has demonstrated that approximately 70% of TB is absorbed after an oral dose (250 mg) (Jensen JC. Clinical & Experimental Dermatology 1989; 14(2): 110-3.), and the maximum plasma concentrations of 0.5-1.5 μg/mL are reached within 2 h. (Humbert U, et. al., Biopharmaceutics & Drug Disposition 1995;16(8):685-94; Kovarik J M, et. al., Antimicrobial Agents & Chemotherapy 1995;39(12):2738-41; Kovarik J M, et. al., British Journal of Dermatology 1992;126(Suppl. 39):8-13.). Another report in a human study showed that the plasma level of TB after daily oral receiving of 250 mg TB for 4 weeks was 1.7+0.77 μg/ml (5.83 EM). (Kovarik J M, et. al., Antimicrobial Agents & Chemotherapy 1995; 39(12):2738-41.). TB is highly lipophilic and keratophilic. It extensively accumulates at the adipose tissues, keratin-rich tissues (such as dermis, epidermis and nail) and other organ tissues. (Faergemann J, et. al., Acta Dermato-Venereologica 1993;73 (4): 305-9; Uosseini-Yeganeh M, and McLachlan AJ. Journal of Pharmaceutical Sciences. 2001;90(11): 1817-28.). The concentrations of the TB in the tissue levels exceeded that of plasma as early as 1 day after stop of medication, and this difference continued to increase until the last day of tissue sampling. Here, we showed that administration of TB at a concentration as low as 1 μM for 3 days arrested the COLO 205 cells at the G0/G1 phase of the cell cycle (FIG. 5). We further demonstrated that the TB-induced cell cycle arrest was irreversible (FIG. 4). Such results implied that continued administration of lower dose TB could reach the therapeutic concentrations in plasma. Importantly, flow cytometry analysis showed that at the doses (10-150 μM) used in our in vitro studies, TB was not cytotoxic for the cultured untransformed human fibroblasts, nor did the TB have any effect on cell proliferation in this culture (FIG. 3F). However, the study of TB effect on the cell growth rate showed that treatment of TB at a concentration of 120 μM for 5 days reduced cell count by 50% in human fibroblasts (FIG. 1F). This might be explained by intracellular accumulation of TB due to a daily change of culture medium for 5 days. Additionally, the dose (50 mg/Kg body weight) used in the present in vivo studies was not cytotoxic for the vital organs.

Although animal studies of TB-induced anti-tumoral action are still ongoing, the findings from the present in vitro and in vivo studies strongly suggest the potential applications of TB in the treatment of human cancer. The universality of TB in the inhibition of cancer cell proliferation would make it a very attractive agent for cancer chemotherapy.

### EXAMPLES

The following examples illustrate methods of preparing, characterizing, and using the composition of the present invention. The examples are in no way intended to limit the scope of the invention.

### Example 1

### Cell Lines and Cell Culture

The HT 29 (p53 mutant) (Niewolik D, et. al., Oncogene 1995;10(5):881-90.) and COLO205 (p53 wild) (HoYS, et. al., Molecular Carcinogenesis 1996;16(1):20-31.) cell lines were isolated from human colon adenocarcinoma (American Type Culture Collection HTB-38 and CCL-222). Hep 3B (p53 partially deleted) (Bressac B, et. al., Proceedings of the National Academy of Sciences of the United States of America 1990;87(5):1973-7.) and Hep G2 (p53 wild) (Bressac B, et. al., Proceedings of the National Academy of Sciences of the United States of America 1990;87(5):1973-7.) cell lines were derived from human hepatocellular carcinoma (ATCC HB-8064 and HB-8065) (Knowles B B, et. al., Science 1980;209(4455):497-9.). Human gingival fibroblasts were harvested by enzymatic dissociation. The HL 60 cell line (p53 null) was derived from human myeloid leukemia cells (59170; American Type Culture Collection). The cell lines were grown in Eagle's minimal essential medium, MEM, (for Hep 3B, Hep G2 and human gingival fibroblasts), or RPMI 1640 (for COLO 205, HT 29 and HL 60 cells) supplemented with 10% fetal calf serum (FCS), 50 μg/mL gentamycin and 0.3 mg/mL glutamine in a humidified incubator (37° C., 5% CO₂). The p53-specific anti-sense (5′-CGGCTCCTCCATGGCAGT-3′) and sense (5′-ACTGCCATGGAGGAGCCG-3′) phosphothioates (S-oligos) were designed as our previously paper described (Chen R J, et. al., Toxicology & Applied Pharmacology 2000;169(2):132-41.), synthesized and purified using high-performance liquid chromatography by Genset.

### Example 2

### Determination of Cell Growth Curve

Human colon cancer, hepatoma, leukemia, and human normal fibroblast cells at a density of 1×10⁴ were plated in 35-mm Petri dishes. TB was added at the indicated doses in 0.05% dimethyl-sulfoxide (DMSO). For control specimens, the same volume of the 0.05% DMSO without TB was added. Media with and without TB were changed daily until cell counting

### Example 3

### Flow Cytometry

The COLO 205 and HT 29 cells were synchronized as previously described. (Chen R J, et. al., Toxicology & Applied Pharmacology 2000;169(2):132-41.). After the cells had grown to 70-80% confluence, they were rendered quiescent by incubation for 24 h in RPMI 1640 containing 0.04% FCS, and challenged with 10% FCS. Then, after release using trypsin-EDTA, they were harvested at various times, washed twice with PBS/0.1% dextrose, and fixed in 70% ethanol at 4° C. Nuclear DNA was stained with a reagent containing propimdiamdiodine (50 DNase-free RNase (2 U/ml) and measured using a fluorescence-activated cell sorter (FACS). The population of nuclei in each phase of the cell cycle was determined using established CellFIT DNA analysis software (Becton Dickenson, San Jose, Calif.).

### Example 4

### Protein Extraction and Western Blot Analysis

As previously described (Chen R J, et. al., Toxicology & Applied Pharmacology 2000;169(2):132-41.), the frozen tumor was pulverized in liquid N₂, and then mixed with lysis buffer (Tris-HCl 0.5 M, pH 6.8, SDS 0.4%). For cell cultures, the cells were seeded onto 150-mm dishes and grown in RPMI 1640 (for COLO 205, HT 29 and HL 60 cells) or MEM (for Hep 3B) supplemented with 10% FCS. After the cells had grown to subconfluence, they were rendered quiescent. The cells were released from quiescence with culture medium supplemented with 10% FCS. TB in 0.05% DMSO or 0.05% DMSO without TB was added to the cells at various concentrations and the mixture was allowed to incubate for 17 h. Western blot analysis was performed as previously described (Chen R J, et. al., Toxicology & Applied Pharmacology 2000;169(2):132-41; Lee W S, et. al., Circulation Research 1998;82(8):845-51.). Immunodetection was carried by probing with proper dilutions of specific antibodies at room temperature for 2 h. Anti-p21/Cip1, anti-p27/Kip1, anti-p53, anti-GAPDH monoclonal antibodies (Santa Cruz, Inc. CA); anti-cyclin B1, D1, and D3, anti-cdk2 and cdk4, and PCNA monclonal antibodies (Transduction Laboratories, Lexington, Ky.) were used at a concentration of 1:1,000 dilution. Anti-cyclin A and E polyclonal antibodies (Transduction, San Diego, Calif.) were used at a concentration of 1:250 dilution. The secondary antibodies, alkaline phosphatase-coupled anti-mouse or anti-rabbit antibody (Jackson, Westgrove Pa.), was incubated at room temperature for 1 h at a concentration of 1:5,000 or 1:1,000 dilution, respectively. The specific protein complexes were identified by incubating with the colorigenic substrates (nitro blue tetrazolium, NBT, and 5-bromo-4-chloro-3-indolyl-phosphate, BCIP; Sigma Chemical Co., St. Louis, Mo.). In each experiment, membranes were also probed with anti-GAPDH antibody to correct for differences in protein loading.

### Example 5

### Immunoprecipitation and Kinase Activity Assay

As previously described (Wu X, et. al., Oncogene 1996;12(7):1397-403.), the TB-treated cells were lysed in Rb lysis buffer, and immunoprecipitated with anti-cdk4 antibody (2 μg). The protein complexes in beads were washed twice with Rb lysis buffer and then once with Rb kinase assay buffer. The level of phosphorylated of Rb (for pRb), histone Hi (for cdk2) and glutathione s-transferase-Rb fusion protein (for cdk4) were measured by incubating the beads with 40 μl of hot Rb kinase solution [0.25 μl (2 μg) of Rb-GST fusion protein, 0.5 μl of (γ-³²P) ATP, 0.5 μl of 0.1 mM ATP and 38.75 μl of Rb kinase buffer] at 37° C. for 30 min, and then stopped by boiling the samples in SDS sample buffer for 5 min. The samples were analyzed by 12% SDS-PAGE, and the gel was then dried and subjected to autoradiography.

### Example 6

### Electrophoretic Mobility Shift Assay (EMSA)

(Somasundaram K, et. al., Nature 1997;389(6647):187-90.) The double-stranded DNA probe used in the experiment contained the p21/Cip1 promoter (5′-CAGGAACAGTCCCAACATGTTGAGC-3′) with p53 consensus binding site. The radio labeled DNA (4 ng, 100,000 cpm) was incubated with nuclear extract in 15 μl of binding buffer (10 mM Tris-HCl, pH 8.0, 1 mM EDTA, 10% glycerol, 200 mM NaCl, and 1 μg probe DNA) on ice for 5 min. The samples were electrophoresed in a 5% polyacrylamide gel, dried on Whatman 3M paper, and then exposed to Fuji x-ray films at −70° C.

### Example 7

### Treatment of COLO 205-Derived Xenografts In Vivo

(Ho Y S, et. al., International Journal of Cancer 2001;91(3):393-401.)

The COLO-205 cells (5×10⁶) in 0.1 ml RPMI 1640 were injected subcutaneously between the scapulae of each nude mouse (purchased from National Science Council animal center, Taipei). After transplantation, the tumor size was measured using calipers and the tumor volume was estimated by the formulae: tumor volume (mm³)=1/2×L×W², where L is the length and W is the width of the tumor. (Oki E, et. al., British Journal of Cancer 1998;78(5):625-30.). Once the tumor reached a volume of 200 mm³, animals received intraperitoneal injections of DMSO (25 μl), TB (50 mg/kg), ND (5 mg/kg), or TB plus ND 3 times per week for 6 weeks.

### Example 8

### DNA Fragmentation Analysis in Tumor Tissues Isolated from the TB-Treated Mouse

The tumor tissues were excised at the end of each experiment.

One part of the tumor tissue was frozen in liquid nitrogen for DNA isolation; the remainder was fixed in 4% paraformaldehyde for detection of apoptotic cells using TdT FragEL™ DNA fragmentation detection kit (Calbiochem Co., Cambridge, Mass. 02142). The DNA isolated from the frozen tumor tissues was used for detection of DNA laddering, a marker of apoptosis, as described previously. (Ho YS, et. al., Toxicology & Applied Pharmacology 1998;153(1):39-47).

### Example 9

### Immunocytochemical Staining Analysis of the Expressions of p53, p21/Cip1 and p27/Kip1 Protein in the COLO 205 Tumor Tissues

The paraffin-embedded blocks were sectioned at 5-7 μm thickness. (Lee W S, et. al., Proceedings of the National Academy of Sciences of the United States of America 1990;87(13):5163-7; Lee W S, et. al., Nature Medicine 1997;3(9):1005-8.). After microwave pretreatment in citrate buffer (pH 6.0) for antigen retrieval, slides were immersed in 0.3% hydrogen peroxide for 20 min to block the endogenous peroxidase activity. After intensive washing with PBS, slides were incubated overnight at 4° C. with the p53, p21/Cip1, and p27/Kip1 antibodies in a dilution of 1:50. After a second incubation with a biotinylated antimouse antibody, slides were incubated with peroxidase-conjugated streptavidin (DAKO LSAB+kit; Dako Corp., Carpinteria, Calif.). Reaction products were visualized by immersing slides in a diaminobenzidine tetrachloride and finally counterstained with hematoxylin.

### Example 10

### Statistics

All data were expressed as the mean value±s.e.mean. Comparisons were subjected to one way analysis of variance (ANOVA) followed by Fisher's least significant difference test. Significance was accepted at P<0.05.

Although the invention has been described with respect to particular embodiments, it will be apparent to those skilled in the art that various changes and modifications can be made without departing from the invention.

1. A method of treating cancer in warm blooded mammals comprising administering to the mammals a therapeutically effective amount of an allylamine derivative in free base form or in pharmaceutically acceptable salt form.

2. The method according to claim 1 wherein said allylamine derivatives are selected from the group consisting of terbinafine, butenafine and naftifine.

3. The method according to claim 1 wherein said allylamine derivative is terbinafine.

4. The method according to claim 1 wherein said amount of said allylamine derivative is more than 1 μM.

5. The method according to claim 1 wherein said amount of said allylamine derivative is about 1 μM to about 150 μM.

6. The method according to claim 1 wherein said cancer includes colon cancer, hepatocellular carcinoma, and leukemia.

7. The method according to claim 1 wherein said cancer includes colon cancer and hepatocellular carcinoma.

8. The method according to claim 1 wherein said cancer is colon cancer.

9. The method according to claim 1 wherein said allylamine derivative is administered to the mammal in combination with an active agent.

10. The method according to claim 9 wherein said active agents are selected from the group consisting of epothillones, albendazole, fenbendazole, nocodazole, parbendazole, mebendazole, oxibendazole, carbendazim, thiabendazole and benzimidazole.

11. The method according to claim 9 wherein said active agent is nocodazole.

12. The method according to claim 9 wherein said allylamine derivative and said active agent are in a proportion of about 10000 to 1.

13. A pharmaceutical composition for treating cancer in warm blooded mammals comprising a therapeutically effective amount of an allylamine derivative and a pharmaceutically acceptable carrier.

14. The composition according to claim 13 wherein said allylamine derivatives are selected from the group consisting of terbinafine, butenafine and naftifine.

15. The composition according to claim 13 wherein said allylamine derivative is terbinafine.

16. The composition according to claim 13 wherein said amount of said allylamine derivative is more than 1 μM.

17. The composition according to claim 13 wherein said amount of said allylamine derivative is about 1 μM to about 150 μM.

18. The composition according to claim 13 wherein said cancer includes colon cancer, hepatocellular carcinoma, and leukemia.

19. The composition according to claim 13 wherein said cancer includes colon cancer and hepatocellular carcinoma.

20. The composition according to claim 13 wherein said cancer is colon cancer.

21. The composition according to claim 13 wherein said allylamine derivative is administered to the mammal in combination with an active agent.

22. The composition according to claim 21 wherein said active agents are selected from the group consisting of epothillones, albendazole, fenbendazole, nocodazole, parbendazole, mebendazole, oxibendazole, carbendazim, thiabendazole and benzimidazole.

23. The composition according to claim 21 wherein said active agent is nocodazole.

24. The composition according to claim 21 wherein said allylamine derivative and said active agent are in a proportion of about 10000 to 1.

25. A method of treating cancer with inducing an anticancer-protein in warm blooded mammals comprising administering to the mammals a therapeutically effective amount of an allylamine derivative in free base form or in pharmaceutically acceptable salt form.

26. The method according to claim 25 wherein said allylamine derivatives are selected from the group consisting of terbinafine, butenafine and naftifine.

27. The method according to claim 25 wherein said allylamine derivative is terbinafine.

28. The method according to claim 25 wherein said amount of said allylamine derivative is more than 1 μM

29. The method according to claim 25 wherein said amount of said allylamine derivative is about 1 μM to about 150 μM.

30. The method according to claim 25 wherein said cancer includes colon cancer, hepatocellular carcinoma, and leukemia.

31. The method according to claim 25 wherein said cancer includes colon cancer or hepatocellular carcinoma.

32. The method according to claim 25 wherein said cancer is colon cancer.

33. The method according to claim 25 wherein said anticaner-protein includes p53, p21/Cip1, and p27/Kip1 protein.

34. The method according to claim 25 wherein said allylamine derivative is administered to the mammal in combination with an active agent.

35. The method according to claim 34 wherein said active agents are selected from the group consisting of epothillones, albendazole, fenbendazole, nocodazole, parbendazole, mebendazole, oxibendazole, carbendazim, thiabendazole and benzimidazole.

36. The method according to claim 34 wherein said active agent is nocodazole.

37. The method according to claim 34 wherein said allylamine derivative and said active agent are in a proportion of about 10000 to 1.

38. A pharmaceutical composition for treating cancer with inducing an anticancer-protein in warm blooded mammals comprising a therapeutically effective amount of an allylamine derivative and a pharmaceutically acceptable carrier.

39. The composition according to claim 38 wherein said allylamine derivatives are selected from the group consisting of terbinafine, butenafine and naftifine.

40. The composition according to claim 38 wherein said allylamine derivative is terbinafine.

41. The composition according to claim 38 wherein a dosage of said allylamine derivative is more than 1 μM

42. The composition according to claim 38 wherein a dosage of said allylamine derivative is about 1 μM to about 150 μM.

43. The composition according to claim 38 wherein said cancer includes colon cancer, hepatocellular carcinoma, and leukemia.

44. The composition according to claim 38 wherein said cancer includes colon cancer or hepatocellular carcinoma.

45. The composition according to claim 38 wherein said cancer is colon cancer.

46. The composition according to claim 38 wherein said anticaner-protein includes p53, p21/Cip1, and p27/Kip1 protein.

47. The composition according to claim 38 wherein said allylamine derivative is administered to the mammal in combination with an active agent.

48. The composition according to claim 47 wherein said active agents are selected from the group consisting of epothillones, albendazole, fenbendazole, nocodazole, parbendazole, mebendazole, oxibendazole, carbendazim, thiabendazole and benzimidazole.

49. The composition according to claim 47 wherein said active agent is nocodazole.

50. The composition according to claim 47 wherein said allylamine derivative and said active agent are in a proportion of about 10000 to 1.

51. A method of treating cancer with inhibiting a cyclin related protein in warm blooded mammals comprising administering to the mammals a therapeutically effective amount of an allylamine derivative in free base form or in pharmaceutically acceptable salt form.

52. The method according to claim 51 wherein said allylamine derivatives are selected from the group consisting of terbinafine, butenafine and naftifine.

53. The method according to claim 51 wherein said allylamine derivative is terbinafine.

54. The method according to claim 51, wherein said amount of said allylamine derivative is more than 1 μM.

55. The method according to claim 51, wherein said amount of said allylamine derivative is about 1 μM to about 150 μM.

56. The method according to claim 51 wherein said cancer includes colon cancer, hepatocellular carcinoma, and leukemia.

57. The method according to claim 51 wherein said cancer includes colon cancer and hepatocellular carcinoma.

58. The method according to claim 51 wherein said cancer is colon cancer.

59. The method according to claim 51 wherein said said cyclin related protein includes cyclin A2, cyclin B, cyclin D3, cyclin-dependent kinase 2, and cyclin-dependent kinase 4.

60. The method according to claim 51 wherein said allylamine derivative is administered to the mammal in combination with an active agent.

61. The method according to claim 60 wherein said active agents are selected from the group consisting of epothillones, albendazole, fenbendazole, nocodazole, parbendazole, mebendazole, oxibendazole, carbendazim, thiabendazole and benzimidazole.

62. The method according to claim 60 wherein said active agent is nocodazole.

63. The method according to claim 60, wherein said allylamine derivative and said active agent are in a proportion of about 10000 to 1.

64. A pharmaceutical composition for treating cancer with inhibiting a cyclin related protein in warm blooded mammals comprising a therapeutically effective amount of an allylamine derivative and a pharmaceutically acceptable carrier.

65. The composition according to claim 64 wherein said allylamine derivatives are selected from the group consisting of terbinafine, butenafine and naftifine.

66. The composition according to claim 64 wherein said allylamine derivative is terbinafine.

67. The composition according to claim 64 wherein said amount of said allylamine derivative is more than 1 μM.

68. The composition according to claim 64 wherein said amount of said allylamine derivative is about 1 μM to about 150 μM.

69. The composition according to claim 64 wherein said cancer includes colon cancer, hepatocellular carcinoma, and leukemia.

70. The composition according to claim 64 wherein said cancer includes colon cancer and hepatocellular carcinoma.

71. The composition according to claim 64 wherein said cancer is colon cancer.

72. The composition according to claim 64 wherein said said cyclin related protein includes cyclin A2, cyclin B, cyclin D3, cyclin-dependent kinase 2, and cyclin-dependent kinase 4.

73. The composition according to claim 64 wherein said allylamine derivative is administered to the mammal in combination with an active agent.

74. The composition according to claim 73 wherein said active agents are selected from the group consisting of epothillones, albendazole, fenbendazole, nocodazole, parbendazole, mebendazole, oxibendazole, carbendazim, thiabendazole and benzimidazole.

75. The composition according to claim 73 wherein said active agent is nocodazole.

76. The composition according to claim 73 wherein said allylamine derivative and said active agent are in a proportion of about 10000 to 1.
